(19) United States
(12) Patent Application Publication
(10) Pub. No.: US 2002/0001806 A1
(43) Pub. Date: Jan. 3, 2002

(54) Caspase-3s splicing variant

(76) Inventors: Yuanhui Huang, Ann Arbor, MI (US); Yi Sun, Ann Arbor, MI (US); Kevin Ka-Wang Wang, Ann Arbor, MI (US)

Correspondence Address: David R. Kurlandsky, Warner-Lambert Company, 2800 Plymouth Road, Ann Arbor, MI, 48105, US

(21) Appl. No.: 09/809,905

(22) Filed: Mar. 16, 2001

Related U.S. Application Data
(60) Provisional application No. 60/204,468, filed on May 16, 2000.

(51) Int. Cl. A61K 48/00; C12Q 1/68; C12N 9/64

(52) U.S. Cl. 435/6; 435/226; 435/325; 514/44; 536/23.2

(57) ABSTRACT

Polynucleotides (sense and antisense) and polypeptides of a novel caspase-3 splicing variant, termed caspase-3s, are described as research reagents, antiapoptotic agents, neuroprotective agents, pharmaceutical compositions, antitumor agents and agents that protect neuronal cells from apoptotic cell death.

### FIELD OF THE INVENTION

The present invention is directed generally to the family of cysteine proteases called caspases, and is more specifically directed at a newly identified splicing variant of caspase-3.

### BACKGROUND OF THE INVENTION

Apoptosis, also referred to as programmed cell death, is generally a programmed process for maintaining homeostasis under physiological conditions and for responding to various stimuli (Thompson, Science 1995;267:1456-1462). Apoptosis is characterized by cell membrane blebbing, cytoplasmic shrinkage, nuclear chromatin condensation, and DNA fragmentation (Wyllie, Int. Rev. Cytol. 1990;68:251-306). Deregulation of apoptosis has been implicated in numerous disease states, including: neurodegenerative disease, graft-versus-host disease, autoimmune disorders as well as cancer (Thornberry et al., Science 1998;281:1312-1316; Thompson, Science 1995;267:1456-1462).

The characteristic manifestations of apoptosis arise from the activation of a highly conserved cell death apparatus within the cell. Central to the cell death apparatus are members of a family of cysteine proteases called caspases. To date there are at least 14 mammalian caspase members, caspase-1 through caspase-14, each having sequence similarity to the cell death gene, ced-3, found in the nematode Caenorhabditis elegan.

Members of the caspase family have several unifying characteristics, including: (1) a conserved QACXG pentapeptide surrounding the protease active site; (2) a conserved polypeptide structure, including a variable length amino terminal prodomain, a centrally located small domain and a carboxy terminal large domain; (3) an expression pattern in the cell whereby the members of the family are expressed as inactive proenzymes that must undergo proteolytic processing before becoming active enzymes; and (4) an activation pattern where proteolyzed caspase subunits associate to form heterodimers having a small subunit and a large subunit (Cryns et al., Genes & Development 1998;12:1551-1570). Heterodimers then associate to form functional tetramers where each tetramer has two independently functioning catalytic sites (Walker et al., Cell 1994;78:343; Rotonda et al., Nature Struct. Biol. 1996;3:619).

The caspase family of enzymes can be loosely broken into a subset of caspases known as upstream “initiators” and a subset of caspases known as downstream “terminators” (Boldin et al., Cell 1996;85:803-815). Initiator caspases include caspases 2, 8, 9, and 10, each of which has been implicated in the activation of one or more of the downstream terminator caspases. The terminator caspases include 3, 6, and 7, and when activated, these caspases help kill the cell by cleaving target substrates at specific sites. Cellular targets of the terminator caspase include: structural molecules like actin filaments, microfilaments and nuclear lamins, DNA repair enzymes like poly (ADP-ribose), polymerase (PARP), and DNA-dependent protein kinase (DNA-PK) and antiapoptotic proteins such as Bcl-2 and Bcl-x_{L}.

Caspase-3 has been directly linked to unscheduled apoptosis in a number of pathological states, such as cerebral ischemia, traumatic brain injury, spinal cord injury, cardiac ischemia (Nicotera et al., Trends Pharmacol. Sci. 1999;20:46-51; Wang, Trends Neurosci. 2000;23:20-26). Experimental results in both cell culture as well as animal models of diseases also show inhibiting caspase-3 and other caspases have a cytoprotective effect against apoptosis, and therefore, such agents might be effective therapeutic agents to combat the above mentioned diseases (Wang, Curr. Opin. Drug Discovery Develop. 1999;2:519-527).

Caspases are mediators of cell death and as such are tightly regulated to prevent improper activation. A component of caspase regulation are a class of proteins and compounds that inhibit caspase activity (Gagliardini et al., Science 1994;263:826-828; Bump et al., Science 1995;269:1885-1888). Caspase inhibitory proteins like the mammalian IAPs are known to confer resistance to apoptotic cell death induced by a broad range of stimuli (Duckett et al., EMBO J. 1996;15:2685-2694). Further, inhibitory compounds like nitric oxide have been shown to inhibit caspase activity by modifying or interfering with the catalytic cysteine residue in the active site of caspase members (Kim et al., J. Biol. Chem. 1997;272:31138-31148). It is likely that other caspase inhibitory products and proteins will be discovered. A better understanding of the caspase inhibitors will allow for improved research into caspase mediated actions within the cell, for improved design of therapeutic drugs involved in chemotherapy and irradiation, as well as in the potential design of genetically engineered inhibitory mechanisms for use in combating aberrant apoptotic disease states. Against this backdrop the present invention has been developed.

### SUMMARY OF THE INVENTION

The present invention provides novel polynucleotides and polypeptides derived therefrom that encode a splicing variant of caspase-3, termed caspase-3s, and that likely act as caspase-3 inhibitors. It is also envisioned that caspase-3s could also inhibit other members of the caspase family.

In one aspect, the present invention provides novel isolated and purified caspase-3s polynucleotides substantially similar to the polynucleotide sequence (SEQ ID No. 1) shown in FIG. 1A. In another embodiment, the present invention comprises a caspase-3s polynucleotide sequence that hybridizes to the polynucleotide sequence (SEQ ID No. 1) shown in FIG. 1A under high stringency hybridization conditions.

Another aspect of the present invention is a recombinant protein that is substantially similar to the caspase-3s polypeptide (SEQ ID No. 2) shown in FIG. 1C. In a further aspect, the present invention is also a substantially purified recombinant protein that is substantially similar to the caspase-3s polypeptide (SEQ ID No. 2) shown in FIG. 1C.

In another aspect, the present invention provides a polynucleotide substantially similar to the caspase-3s polynucleotide sequence (SEQ ID No. 1) shown in FIG. 1A subcloned into an extra-chromosomal vector. In a further aspect, the present invention provides recombinant host cells that are transfected with a recombinant polynucleotide comprising a polynucleotide substantially similar to the caspase-3s polynucleotide sequence (SEQ ID No. 1) shown in FIG. 1A subcloned into an extra-chromosomal vector.

Another aspect of the invention is a method for inducing neuroprotection in a cell (particaulary in central neurons) or organ (such as brain) by administering a cytoprotective dose of caspase-3s to the cell or the whole organism.

Another aspect of the invention is a pharmaceutical composition having a substantially purified caspase-3s polynucleotide and a pharmaceutically acceptable carrier.

Another aspect of the invention is a method for inducing apoptosis of a tumor cell by making a recombinant vector (such as adenovirus) that expresses an antisense caspase-3s mRNA and administering the recombinant vector to the tumor cell.

Another aspect of the invention is a method for inhibiting neuronal cell death (such as induced by stroke or neurodegenerative disorders) by making a recombinant vector (such as adenovirus) that expresses a caspase-3s polypeptide and administering the recombinant vector to the neuronal cells or whole brain.

### BRIEF DESCRIPTION OF THE DRAWINGS

FIG. 1A shows the cDNA sequence of caspase-3 and caspase-3s.

FIG. 1B is a schematic representation of the structural domains of caspase-3 and caspase-3s.

FIG. 1C shows the deduced amino acid sequence of caspase-3 and caspase-3s.

FIG. 1D is a schematic representation of caspase-3 and caspase-3s illustrating the location of the QACRG pentapeptide of caspase-3.

FIG. 2A is an autoradiogram of a RT-PCR (reverse transcription-polymerase chain reaction)/Southern blot showing the distribution of caspase-3 and caspase-3s found in various human tissues.

FIG. 2B and 2C are autoradiograms of RT-PCR/Southern blots showing the distribution of caspase-3 and caspase-3s in several human cancer cell lines.

FIG. 3A is an autoradiogram of a western blot showing the in vitro expression of caspase-3 (lane 1) and caspase-3s (lane 2) that was subcloned in a pcDNA3 vector and expressed in a TNT Coupled Reticulocyte Lysate System.

FIG. 3B is an autoradiogram of a western blot showing in vivo expression of caspase-3 (lane 1) and caspase-3s (lane 2) that was subcloned into a pcDNA3 vector and transfected into transformed human embryonic kidney cells.

FIG. 4 is a graph showing that caspase-3s inhibits apoptotic cell death induced by proteasome inhibitor I (Z-Ile-Glu(otBu)-Ala-Leu-CHO).

FIG. 5A. Caspase-3s inhibit cells death induced by proteasome inhibitor I (Z-Ile-Glu(otBu)-Ala-Leu-CHO). pcDNA3 vector, caspase-3 and caspase-3s were transfected into 293 cells. The transfected cells were then treated with 5 μM of IGAL or 0.5 μM staurosporine. DNA fragmentation was measured by ELISA after 16 to 20 hours of treatment. In IGAL treated cells, transfection of caspase-3s significantly (# p<0.05) inhibited DNA fragmentation as compared to the vector control and caspase-3 transfected cells after the treatment (n=4). For untreated cells, transfection of caspase-3 significantly increased DNA fragmentation as compared to both vector and caspase-3s transfected cells (*p<0.05). Caspase-3s had no significant effect on apoptosis induced by staurosporine treatment. B. Caspase-3s partially inhibited PARP cleavage after treatment with proteasome inhibitor as compared to the vector control. 293 cells were transfected with caspase-3, caspase-3s, or pcDNA3 vector followed by IGAL treatment for 16 hours or left untreated (control). PARP expression is measured by Western blot. C. Densitometric quantification of PARP breakdown product (85 kDa) from panel B (n=8). The results were normalized to vector control and presented as percentage. The PARP breakdown product in caspase-3s transfected cells was significantly decreased while transfection of caspase-3 increased PARP cleavage. Both were compared to the vector control.

FIG. 6. A. 293 cells were transfected with flag-tagged caspase-3, flag-tagged caspase-3s or pcDNA3 vector, followed by IGAL treatment for 16 hours or left untreated (control). Fifty microgram of total cellular protein was assayed for caspase-3 and caspase-3s expression using antibody specific for flag-tag. The asterisk (*) indicates the N-terminal flag-tag containing activated capase-3 (20 kDa). The uppermost and lowest short arrow denote the two additional bands, 40 kDa and 14 kDa, respectively. B. Protease inhibitor treatment stabilized endogenous caspase-3 expression. 293 cells were treated with 5 μM IGAL for 48 hours. Total cell lysates were isolated and assayed for caspase-3 expression by Western blotting. The blot was probed with anticaspase-3 antibody (Pharmingen).

### DETAILED DESCRIPTION OF THE INVENTION

Definitions

The following definitions are provided to facilitate understanding of certain terms used frequently herein and are not meant to limit the scope of the present disclosure.

The term host cell or host cells refers to cells established in ex vivo culture. It is a characteristic of host cells discussed in the present disclosure that they be capable of expressing either caspase-3 or caspase-3s. Examples of suitable host cells useful for aspects of the present invention include insect and mammalian cells. Specific examples of such cells include SF9 insect cells (Summers and Smith, Texas Agriculture Experiment Station Bulletin, 1987:1555), human embyonic kidney cells (293 cells), Chinese hamster ovary (CHO) cells (Puck et al., Proc. Natl. Acad. Sci. USA 1958;60:1275-1281), human cervical carcinoma cells (HELA) (ATCC CCL 2), human liver cells (Hep G2) (ATCC HB8065), human breast cancer cells (MCF-7) (ATCC HTB22), human neuroblastoma cells (SY5Y) (a gift from Dr. Steve Fisher at University of Michigan), and human colon carcinoma cells (DLD-1) (ATCC CCL 221), etc.

The term “nucleic acid sequence” refers to the order or sequence of deoxyribonucleotides along a strand of deoxyribonucleic acid. The order of these deoxyribonucleotides determines the order of amino acids along a polypeptide chain. The deoxyribonucleotide sequence thus codes for the amino acid sequence.

The term “polynucleotide” refers to a linear sequence of nucleotides. The nucleotides are either a linear sequence of polyribonucleotides or polydeoxyribonucleotides, or a mixture of both. Examples of polynucleotides in the context of the present invention include—single and double stranded DNA, single and double stranded RNA, and hybrid molecules that have both mixtures of single and double stranded DNA and RNA. Further, the polynucleotides of the present invention may have one or more modified nucleotides.

As used herein, the term splicing variant or variant means a polynucleotide that differs from a reference polynucleotide. Variants can include nucleotide changes that result in amino acid substitutions, deletions, fusions, and truncations in the resulting variant polypeptide when compared to the reference polypeptide.

As used herein the term variant may also refer to a variant polypeptide which may differ from a reference polypeptide in amino acid sequence by one or more substitutions, additions, deletions, fusions, and/or truncations.

As used herein, protein, peptide, and polypeptide are used interchangeably to denote an amino acid polymer or a set of two or more interacting or bound amino acid polymers.

As used herein, a “substantially purified” protein is one that is free from at least 5% to 10% of the contaminating protein.

As used herein, amino acids mean any of the 20 gene encoded amino acids as well as any modified amino acid sequences. Modifications may include natural processes such as posttranslational processing, or may include chemical modifications which are known in the art. Modifications include but are not limited to: phosphorylation, ubiquitination, acetylation, amidation, glycosylatioin, covalent attachment of flavin, ADP-ribosylation, cross linking, iodination, methylation, etc.

The term “tumor cell” within the context of the present invention is used synonymously with cancer cell and means a cell that has lost, in some manner, its ability to respond to normal growth signals, ie, is undergoing abnormally regulated growth.

The term vector, extra-chromosomal vector or expression vector, refers to a first piece of DNA, usually double-stranded, which may have inserted into it a second piece of DNA, for example a piece of foreign DNA like the cDNA of caspase-3s. Foreign DNA is defined as heterologous DNA, which is DNA that may or may not be naturally found in the host cell and includes additional copies of nucleic acid sequences naturally present in the host genome. The vector transports the foreign DNA into a suitable host cell. Once in the host cell the vector may be capable of integrating into the host cell chromosomes. The vector may also contain the necessary elements to select cells containing the integrated DNA as well as elements to promote transcription of mRNA from the transfected DNA. Examples of vectors within the scope of the present invention include, but are not limited to, plasmids, bacteriophages, cosmids, retroviruses, and artificial chromosomes.

The term “expression” refers to transcription and translation occurring within a host cell. The level of expression of a DNA molecule in a host cell may be determined on the basis of either the amount of corresponding mRNA that is present within the cell or the amount of DNA molecule encoded protein produced by the host cell (Sambrook et al., Molecular cloning: A Laboratory Manual 1989:18.1-18.88).

Modes for Carrying Out the Invention

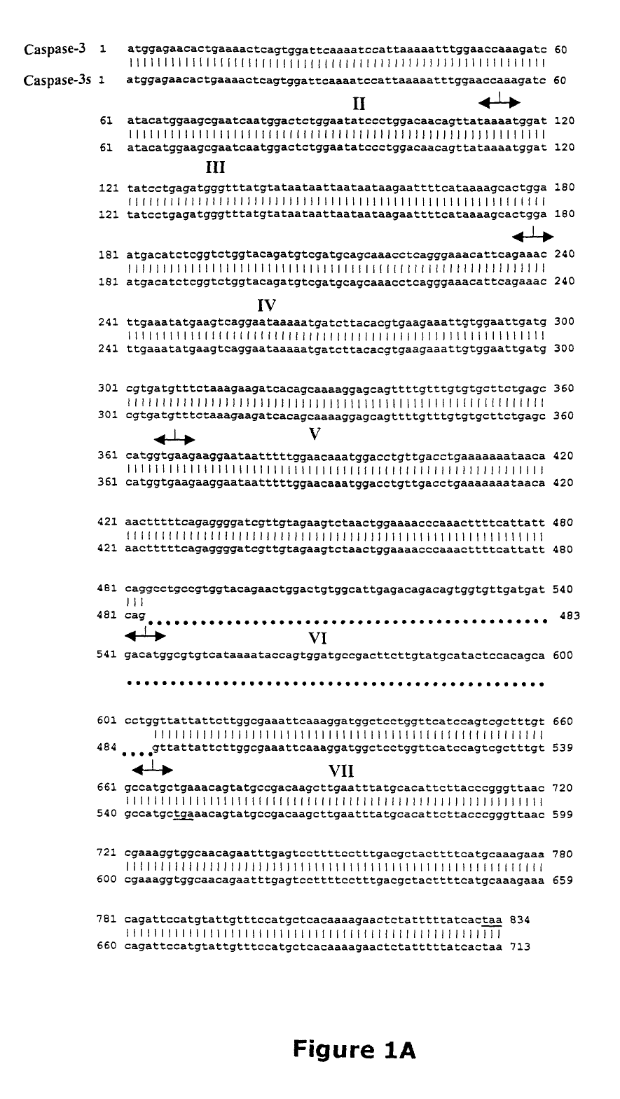

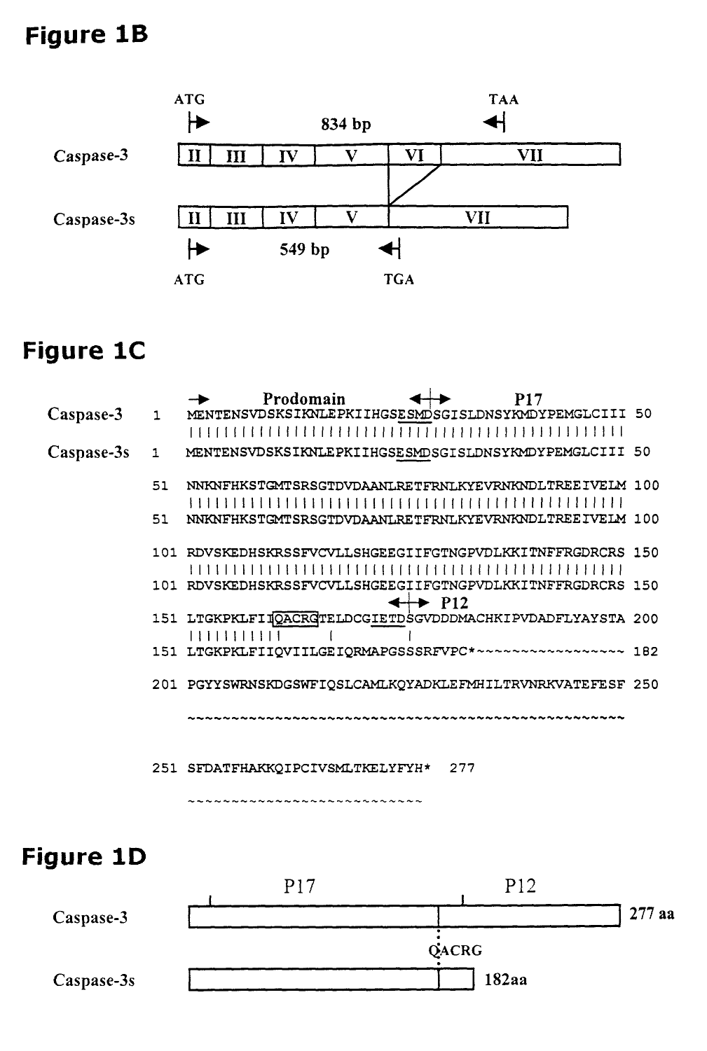

The present invention is based upon, among other things, the discovery of a novel splicing variant of caspase-3, termed caspase-3s. In particular, the present invention is related to the caspase-3s polynucleotide (SEQ ID No. 1) as shown in FIG. 1A and the caspase-3s polypeptide (SEQ ID No. 2) as shown in FIG. 1C. The isolation of such polynucleotides and polypeptides permits a more detailed analysis of caspase-3 molecular actions as well as provides laboratory tools useful in identifying the mechanisms of caspase-3 mediated apoptosis and in enabling improved design of therapeutic drugs that regulate apoptosis.

It is believed from an analysis of the caspase-3s polynucleotide sequence (SEQ ID No. 1) and corresponding polypeptide sequence (SEQ ID No. 2) that caspase-3s is a caspase-3, and possibly a general caspase family member, inhibitor. It might do so by either (i) forming inactive heterodimer with endogenous full length caspase-3 or by (ii) binding and forming stable complex with the initiator caspases (caspase-8, -9, and -10).

Within this application, unless otherwise stated, the techniques utilized may be found in any of several well-known references such as (Sambrook et al., Molecular cloning: A Laboratory Manual, 1989), Gene Expression Technology (Methods in Enzymology, 1989:185, edited by D. Goeddel, Academic Press, San Diego, Calif., 1991), “Guide to Protein Purification” in Methods in Enzymology (M. P. Deutshcer, 3d., Academic Press, Inc., 1990), PCR Protocols: A Guide to Methods and applications (Innis et al., Academic Press, San Diego, Calif., 1990), Culture of Animal Cells: A Manual of Basic Technique, 2^{nd} ed. (Freshney R. I., Liss, Inc., New York, N.Y., 1987), and Gene Transfer and Expression Protocols, year: 109-128, ed. E. J. Murray, The Humana Press Inc., Clifton, N.J.).

Polynucleotides

In one aspect of the present invention, there is provided a novel isolated and purified polynucleotide sequence of caspase-3s (SEQ ID No. 1) as is shown in FIG. 1A. Further, the present invention comprises polynucleotide sequences substantially similar to (SEQ ID No. 1), where the caspase-3s sequence of SEQ ID No.1 can include deletions, substitutions or additions to the polynucleotide so long as the polynucleotide maintains caspase-3s function.

Polynucleotides of the present invention can include RNA, such as mRNA, or can be DNA obtained by cloning or produced by synthetic techniques. The cDNA sequence (SEQ ID No. 1) shown in FIG. 1A may contain additional DNA sequences, such as those that encode additional amino acids which provide additional functionalities. Examples of additional DNA sequence are marker sequence that facilitate purification of fused polypeptides and detection sequence that facilitate detection of fused polypeptides. A specific example of one such additional sequence is the hexahistidine peptide tag for convenient purification of the tag containing fusion protein (Gentz et al., Proc. Natl. Acad. Sci., USA 1989;86:821-824).

Another embodiment of the present invention is a nucleotide sequence that hybridizes to the caspase-3s sequence (SEQ ID No. 1) shown in FIG. 1A under high stringency hybridization conditions. High stringency conditions refers to hybridization at 65° C. in a low salt hybridization buffer to the probe of interest at 2×10⁸ cmp/μg for between about 8 hours to 24 hours, followed by washing in 1% SDS, 20 mM phosphate buffer and 1 mM EDTA at 65° C., for between 30 and 60 minutes. The low salt hybridization buffer may include 0.5% to 10% SDS and 0.05 M sodium phosphate. The DNA sequences of this embodiment can be used to direct expression of the caspase-3s protein and for mutational analysis of the caspase-3s protein function, and are isolated via hybridization as described.

The present invention also includes polynucleotide fragments, analogs and derivatives of the sequence of SEQ ID No. 1 and shown in FIG. 1A. A fragment, analog or derivative may be made by mutagenesis techniques or other methods known to the art. Additionally, the polynucleotide fragments, analogs and derivatives may include substitutions, deletions, or additions that involve one or more nucleotides.

Another embodiment of the present invention are polynucleotides that are at least 75% identical to the polynucleotide sequence for caspase-3s (SEQ ID No. 1) shown in FIG. 1A. Further, preferred embodiments are between 80% and 95% identical to the caspase-3s cDNA and highly preferred embodiments are between 95% and 99% identical.

The polynucleotides of the present invention may also be used as research reagents and materials in the study of apoptosis. Additionally, the polynucleotides may be used in the treatment of human disease—such as in gene therapy techniques on introducing the caspase-3s polynucleotide (in an antisense orientation) into tumors or into individual cancer cells or introducing the caspase-3s polynucleotide (in a sense orientation) into brain or into individual neurons.

Polypeptides

Another aspect of the present invention is a recombinant protein that is substantially similar to the caspase-3s polypeptide (SEQ ID No. 2) shown in FIG. 1C. Aspects of the present invention also include naturally occurring caspase-3s proteins and synthetic caspase-3s proteins. As used herein substantially similar includes deletions, substitutions, and additions to the sequence of SEQ ID No. 2 introduced by any in vitro means. Altered sequences according to the invention can be identified in a routine manner by those skilled in the art.

Another aspect of the present invention is a substantially purified recombinant protein that is substantially similar to the caspase-3s polypeptide of SEQ ID No. 2.

A further aspect of the present invention is the use of the recombinant protein(s) that are substantially similar to the caspase-3s polypeptide (SEQ ID No. 1) and shown in FIG. 1C as novel tools for use in in vitro assays and as components for pharmaceutical compositions.

Another variant of the present invention relates to fragments, analogs, and derivatives of the caspase-3s polypeptide (SEQ ID No. 2) shown in FIG. 1C. These caspase-3s fragments, analogs, and derivatives must retain the biologic functions and activities of the caspase-3s polypeptide (SEQ ID No. 2) shown in FIG. 1C.

Another variant of the present invention relates to a polypeptide that has conserved amino acid substitutions in the caspase-3s polypeptide (SEQ ID No. 2) shown in FIG. 1C. The conserved amino acid substitutions are those that substitute one amino acid for an amino acid of like characteristic. Thus, for example, the substitution of an Asp for a Glu, as both residues are acidic residues.

Finally, the polypeptides of the present invention also include amino acid sequences that are at least 70% similar to the sequence of caspase-3s (SEQ ID No. 2) as shown in FIG. 1C.

Vectors and Host Cells

In another aspect of the present invention, novel polynucleotides substantially similar to the caspase-3s polynucleotide sequence (SEQ ID No. 1) are subcloned into an extra-chromosomal vector. The subcloned polynucleotide(s) may be joined to a vector having a cis-acting or regulatory element for increased propagation in a host cell (note that the trans-acting factors involved are supplied to the host, supplied by a second vector or supplied by the vector itself upon introduction into the host). This aspect of the invention allows for the in vivo and in vitro expression of the caspase-3s polynucleotide, thus permitting an analysis of caspase-3s structure and function. Several vectors that can be used in the context of this invention include: PcDNA3 vector (Invitrogen), vectors having the T3 and T7 polymerase promoters, vectors having the SV40 promoter or the CMV promoter, or any other promoter that either can direct expression of a polypeptide off a polynucleotide, or that one wishes to test for the ability to direct expression of a polypeptide off a polynucleotide.

In a further aspect of the present invention, host cells can be genetically engineered to incorporate the polynucleotides of the present invention and to express the polypeptides of the present invention. Techniques required for this aspect of the invention are well known in the art (Sambrook et al., Molecular Cloning: A Laboratory Manual, 2^{nd} ed., Cold Spring Harbor Press, 1989) and can include calcium phosphate transfection, dextran sulfate transfection, electroporation, lipofection, and viral infection (see Graham and van der Eb, Virology 1978;52:456-457; Chisholm et al., DNA Cloning IV: A Practical Approach, Mammalian Systems, Glover and Hanes, eds., 1995:1-41; Andreason, J. Tisss. Cult. Meth. 1993;15:56-62).

The host cells of the present invention may be of any type, including, but not limited to, noneukaryotic and eukaryotic cells. Host cells are cultured using standard tissue culture techniques in conventional media as is well-known in the art. The level of expression of the caspase-3s cDNA introduced into a host cell of the invention depends on multiple factors, including gene copy number, efficiency of transcription, messenger RNA processing, stability, and translation efficiency. Accordingly, high level expression of a desired caspase-3s polypeptide according to the present invention will typically involve optimizing one or more of those factors.

Another aspect of the present invention is a method for inhibiting the growth of mammalian and/or nonmammalian tumor cells, which involves introducing into the tumor cells an expression vector having a substantially similar sequence to the caspase-3s polynucleotide sequence (SEQ ID No. 1).

Neuron Protection

The caspase-3s polypeptides of the invention are effective protective agents, for example, protecting neuronal cells from apoptosis. In the methods of the invention, the protective effects of caspase-3s are achieved by treating, such as neuronal cells, with micromolar amounts of the protective compound.

The present invention also includes polynucleotide fragments, analogs, and derivatives of caspase-3s as used as cytotoxic compounds. A fragment, analog, or derivative may be made by mutagenesis techniques or other methods known to the art. Additionally, the polynucleotide fragments, analogs, and derivatives may include substitutions, deletions, or additions that involve one or more nucleotides.

Tumor Treatment

The caspase-3s polynucleotide sequence (SEQ ID No. 1) can also be used in its antisense orientation in methods of tumor treatment, for example, by administering to a subject an antisense caspase-3s polynucleotide in order to achieve an inhibition of tumor cell growth, a killing of tumor cells, induced apoptosis, and/or increased patient survival time.

The present invention also includes polynucleotide fragments, analogs, and derivatives of caspase-3s as used in tumor treatment. A fragment, analog, or derivative may be made by mutagenesis or random fragmentation techniques, restriction enzyme digestion, or other methods known to the art. Additionally, the polynucleotide fragments, analogs, and derivatives may include substitutions, deletions, or additions that involve one or more nucleotides.

Caspase-3s polynucleotides are suitable for use in mammals. As used herein, “mammals” means any class of higher vertebrates that nourish their young with milk secreted by mammary glands, including, for example, humans, rabbits, and monkeys.

Administration Methods

Polynucleotides substantially similar to the caspase-3s polynucleotide sequence (SEQ ID No. 1) can be formulated as pharmaceutical compositions and administered to a mammalian host, including a human patient, in a variety of forms adapted to the chosen route of administration. The compounds are preferably administered in combination with a pharmaceutically acceptable carrier, and may be combined with or conjugated to specific delivery agents, including targeting antibodies and/or cytokines.

Caspase-3s can be administered by known techniques, such as orally, parentally (including subcutaneous injection, intravenous, intramuscular, intrasternal, or infusion techniques), by inhalation spray, topically, by absorption through a mucous membrane, or rectally, in dosage unit formulations containing conventional nontoxic pharmaceutically acceptable carriers, adjuvants or vehicles. Pharmaceutical compositions of the invention can be in the form of suspensions or tablets suitable for oral administration, nasal sprays, creams, sterile injectable preparations, such as sterile injectable aqueous or oleagenous suspensions or suppositories.

For oral administration as a suspension, the compositions can be prepared according to techniques well-known in the art of pharmaceutical formulation. The compositions can contain microcrystalline cellulose for imparting bulk, alginic acid, or sodium alginate as a suspending agent, methylcellulose as a viscosity enhancer, and sweeteners or flavoring agents. As immediate release tablets, the compositions can contain microcrystalline cellulose, starch, magnesium stearate and lactose or other excipients, binders, extenders, disintegrants, diluents, and lubricants known in the art.

For administration by inhalation or aerosol, the compositions can be prepared according to techniques well-known in the art of pharmaceutical formulation. The compositions can be prepared as solutions in saline, using benzyl alcohol or other suitable preservatives, absorption promoters to enhance bioavailability, fluorocarbons, or other solubilizing or dispersing agents known in the art.

For administration as injectable solutions or suspensions, the compositions can be formulated according to techniques well-known in the art, using suitable dispersing or wetting and suspending agents, such as sterile oils, including synthetic mono- or diglycerides, and fatty acids, including oleic acid.

For rectal administration as suppositories, the compositions can be prepared by mixing with a suitable non-irritating excipient, such as cocoa butter, synthetic glyceride esters, or polyethylene glycols, which are solid at ambient temperatures, but liquefy or dissolve in the rectal cavity to release the drug.

Preferred administration routes include orally, parenterally, as well as intravenous, intramuscular or subcutaneous routes. More preferably, the compounds of the present invention are administered parenterally, ie, intravenously or intraperitoneally, by infusion or injection. In one embodiment of the invention, the compounds may be administered directly to a tumor by tumor injection; or by systemic delivery by intravenous injection.

Solutions or suspensions of the compounds can be prepared in water, isotonic saline (PBS), and optionally mixed with a nontoxic surfactant. Dispersions may also be prepared in glycerol, liquid polyethylene, glycols, DNA, vegetable oils, triacetin, and mixtures thereof. Under ordinary conditions of storage and use, these preparations may contain a preservative to prevent the growth of microorganisms.

The pharmaceutical dosage form suitable for injection or infusion use can include sterile, aqueous solutions or dispersions, or sterile powders comprising an active ingredient which are adapted for the extemporaneous preparation of sterile injectable or infusible solutions or dispersions. In all cases, the ultimate dosage form should be sterile, fluid, and stable under the conditions of manufacture and storage. The liquid carrier or vehicle can be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. The proper fluidity can be maintained, for example, by the formation of liposomes, by the maintenance of the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption-for example, aluminum monosterate hydrogels and gelatin.

Sterile injectable solutions are prepared by incorporating the compounds in the required amount in the appropriate solvent with various other ingredients as enumerated above and, as required, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

A proteasome inhibitor, such as proteasome inhibitor I (Z-Ile-Glu(otBu)-Ala-Leu-CHO) (IGAL), can be coadministered with the caspase-3s. Proteasome inhibitors have been shown (see below) to increase the half-life of caspase-3s, thereby enhancing the utility of caspase-3s.

### EXAMPLES

The following examples are provided to illustrate the invention only, and should not be construed as limiting the scope of the invention. All literature citations herein are expressly incorporated by reference.

### Example 1

Cloning of Caspase-3 and Caspase-3s

Reverse-transcriptase polymerase chain reaction (RT-PCR) was carried out by reversibly transcribing total RNA, which was isolated from human colon carcinoma DLD-1 cells, into first strand cDNA, in a reaction mixture containing 5 to 10 μg of total RNA, 1 μg of oligo dT (Pharmacia), 40 units of Rnasin (Promega), 45 units of AMV reverse transcriptase (Seikagaku America, Inc.), and 1×RT buffer (100 mM Tris, 80 mM KCl, 10 mM MgCl₂ and 1 mM dNTP). The reaction mixture was incubated at 42° C. for 90 minutes. Sodium hydroxide was added to samples to a final concentration of 0.5N and incubated at 70° for an additional 30 minutes. DNA was precipitated in the presence of 0.1 volume of 3 M NaAC and 2.5 volumes of cold ethanol at −70° C. for 1 hour. Precipitated DNA was centrifuged at 15,000 rpm for 15 minutes at 4° C. and washed with 70% ethanol, air dried and dissolved in 50 μL of ddH₂O.

The resultant cDNA was subjected to PCR using a pair of primers having caspase-3 specific sequence, the caspase-3 sequence is indicated by underlining_{—}

5′-primer: CGCGGATCCGCCACCATGGACTACAAGGACGACGATGACAAGGAGAACACTGAAAACTCAGTG (SEQ ID No. 3)

3′-primer: CCGCTCGAGTTAGTGATAAAAATAGAGTTCTTTTGTGAGC (SEQ ID No. 4).

A BamH 1 restriction site and Flag tag were added to the 5′-primer and a Xho 1 restriction site was added to the 3′-primer. Additionally, the cDNA was subjected to PCR using a pair of primers having caspase-3s specific sequence where the 5′-primer was identical to caspase-3 primer above and where the 3′-primer has caspase-3s specific sequence, as indicated by underlining-

5′-primer (same as above): CGCGGATCCGCCACCATGGACTACAAGGACGACGATGACAAGGAGAA CACTGAAAACTCAGTG (SEQ ID No. 3)

3′-primer: CCGCTCGAGTCAGCATGGCACAAAGCGACTGGATGAAC (SEQ ID No.5).

PCR reactions were carried out for 60 s at 95° C., 60 s at 55° C., and 120 s at 72° C. for 35 cycles. PCR products were restricted with BamH 1 and Xho 1 endonuclease, gel purified, extracted, and subcloned into PcDNA vector (Invitrogen).

Results:

Caspase-3 and caspase-3s cDNA sequence are shown in FIG. 1A. The overall sequence between caspase-3 and caspase-3s are identical except for a 121 nucleotide gap between nucleotides 484 and 604 located in the caspase-3s cDNA.

Referring to FIG. 1C, a comparison of the deduced caspase-3 and caspase-3s amino acid sequences are shown. The two polypeptide chains have identical amino acid sequences to the point of the 121 nucleotide gap at which point the caspase-3s polypeptide diverges from the caspase-3 polypeptide to form a shortened truncated protein that terminates at amino acid 182.

The data indicates that caspase-3s expresses a protein product having identical sequence similarity to caspase-3 for its first 161 amino acids. However, the caspase-3s protein terminates at amino acid 182 because of a TGA sequence that results from the shifted reading frame found after the 121 nucleotide gap in the caspase-3s cDNA. FIGS. 1B and 1D schematically illustrate the caspase-3 and caspase-3s protein structure side-by-side.

The cloning and sequencing data of caspase-3s indicates that caspase-3s is a C-terminal truncated version of caspase-3 which does not have a protease active site, nor an activating cleavage site. This data suggests that caspase-3s should be able to form inactive heterodimers with caspase-3 thereby reducing the pool of active caspase-3 proteins available in the cell during a proapoptotic event.

### Example 2

Distribution of Caspase-3s in Normal Tissue

RT-PCR/Southern blot analysis was carried out to examine caspase-3s distribution in normal tissue using a standard protocol (Sambrook et al., Molecular cloning: A laboratory manual). First-strand cDNA generated by RT reaction (Multiple Tissue cDNA Panels, Clontech) from various human tissues were PCR amplified. The PCR products were resolved in a 1% agarose gel, and the gels were soaked in denaturing solution (0.2N NaOH, 0.6 M NaCl) at room temperature for 30 minutes, followed by neutralization with neutralization buffer (0.5 M Tris, pH 7.5, 1.5 M NaCl) for 30 minutes. PCR products were transferred to a nylon membrane (Life Science Products, Inc.) with 10×SSC buffer and were cross-linked by exposure to UV light for 105 seconds. Southern hybridization was performed by first incubating the nylon membrane in prehybridization solution at 60° C. for at least 1 hour, followed by addition of a β-3²P labeled caspase-3s probe having a sequence that corresponds to the entire open reading frame of the caspase-3 coding sequence. The probe was engineered to hybridize to both the caspase-3 and caspase-3s coding sequence (see FIG. 1A). The nylon membrane was incubated with the caspase-3 variant probe for 12 to 24 hours at 60° C., washed several times with washing buffer, and exposed to film for 2 to 24 hours at −80° C.

Results:

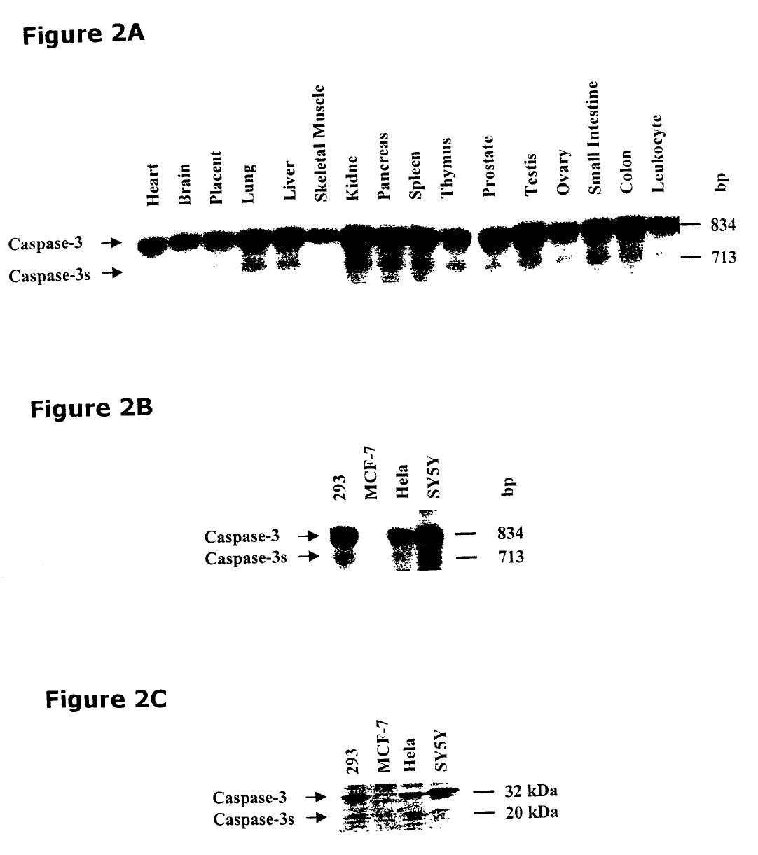

Referring to FIG. 2A, the caspase-3s variant probe hybridized to a 834 bp and 713 bp cDNA species. The 834 bp species corresponds to caspase-3. As expected, the wildtype caspase-3 species was detected in all tissues examined including heart, brain, placenta, lung, liver, skeletal muscle, kidney, pancreas, spleen, thymus, prostate, testis, ovary, small intestine, colon, and peripheral blood leukocytes. A 713 bp cDNA species corresponding to the expected size of the caspase-3s cDNA was detected at lower levels in all tissues examined with the possible exception of skeletal muscle, and with minimal levels found in the heart, brain, and peripheral blood leukocytes.

The data demonstrate that the splicing variant caspase-3s is normally expressed in a diverse number of tissues. The distribution and expression level of caspase-3s corresponds with caspase-3s being expressed in normal tissues but having reduced or eliminated expression in tissues having little or no growth potential like the brain, heart, placenta, peripheral blood leukocytes, and skeletal muscle.

### Example 3

Distribution of Caspase-3s in Cultured Cancer Cell Lines

The materials and methods discussed in Example 3 were used to examine caspase-3s distribution in several established human cancer cell lines, including human embryonic kidney 293 cells, human breast cancer MCF-7 cells, human cervical carcinoma Hela cells, and human neuroblastoma SY5Y cells.

Results:

Referring to FIGS. 2B and 2C, the caspase-3 variant probe hybridized to the 834 bp and 713 bp cDNA species that correspond to caspase-3 and caspase-3s respectively in the 293 cells, Hela cells and SY5Y cells. However, no caspase-3 or caspase-3s signal was detected in MCF-7 cells.

Interestingly, failure to express caspase-3 in MCF-7 cells may indicate one factor involved in the loss of regulatory control in these breast cancer cells. Additionally, the data shown in FIG. 2B suggests that caspase-3s is expressed at greater levels in the neuroblastoma cell line than in normal brain tissue (compare FIGS. 2A and 2B). This data suggests a potential role for caspase-3s in the development of brain tumors as well as in other types of cancer.

### Example 4

In Vitro Expression of Caspase-3s

Full length caspase-3 and caspase-3s cDNA (FIG. 1A) were constructed into pcDNA3 vectors (Invitrogen) and expressed in a TNT Coupled Reticulocyte Lysate System (Promega) according to the protocol provided by the supplier. Protein products were separated on standard 4% to 20% sodium dodecyl sulfate (SDS)-polyacrylamide gels and analyzed by ECL western blotting using an anti-flag antibody (Sigma) and ECL western blotting detection reagents (Amersham).

Results:

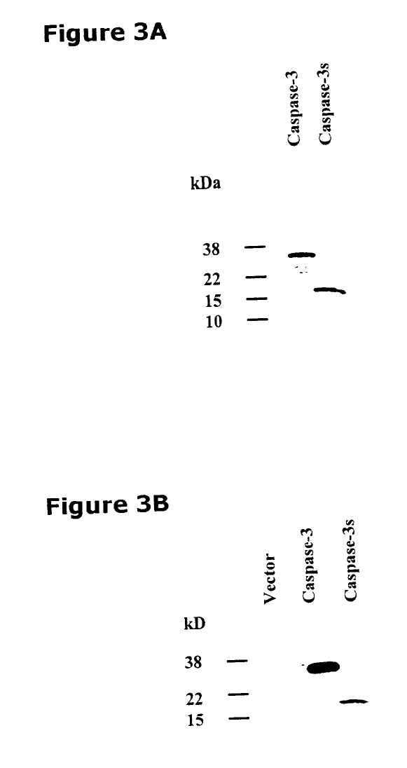

Data shown in FIG. 3A shows that caspase-3 and caspase-3s are expressed at high levels in the in vitro system. Protein products correspond to the expected molecular weight: 32 kDa for caspase-3 and -20 kDa for caspase 3s.

### Example 5

In Vivo Expression of Caspase-3s

Full length caspase-3 and caspase-3s cDNA (FIG. 1A) were constructed into pcDNA3 vectors (Invitrogen) and transiently transfected into a transformed human embryonic kidney cell line, 293 cells. Transfection technique was as previously described (Sun et al., Oncogene 1997;14:385-393). Protein products of the constructs were separated on standard 4% to 20% SDS-polyacrylamide gels and analyzed using anti-flag antibody and ECL western blotting detection reagents.

Results:

Data shown in FIG. 3B shows that caspase-3 (Lane 1) and caspase-3s (Lane 2) are expressed at high levels in the in vivo system. Protein products correspond to the expected molecular weights for each expressed protein.

### Example 6

Caspase-3s Protects Cells From Apoptosis Induced By Proteasome Inhibitors

The following experiments were performed to test the hypothesis that caspase-3s acts as a dominant negative isoform of caspase-3. The pcDNA empty vector, caspase-3, and caspase-3s constructs were transfected into 293 cells using the method described above (Sun et al., Supra., 1997). After 6 to 7 hours transfection, transfection medium was replaced with fresh medium containing 5 μM of proteasome inhibitor I (Z-Ile-Glu(otBu)-Ala-Leu-CHO) (Calbiochem). Cells were then incubated for 12 hours. Twenty-five microliter of the culture medium was subjected to cytotoxicity assay using the Cytotox 96 colorimetric LDH assay kit (Promega) according to manufacturer's instruction. Statistics were performed with t-test using Microsoft Excel analysis tools.

Caspase-3s inhibits cell death induced by the apoptosis inducing agent Proteasome Inhibitor I (Z-Ile-Glu(otBu)-Ala-Leu-CHO). For control cells, lactate dehydrogenase (LDH) release in caspase-3 transfected cells was compared with both vector and caspase-3s transfected cells. For proteasome inhibitor treated cells, LDH release was compared between vector and caspase-3s transfected cells.

Results:

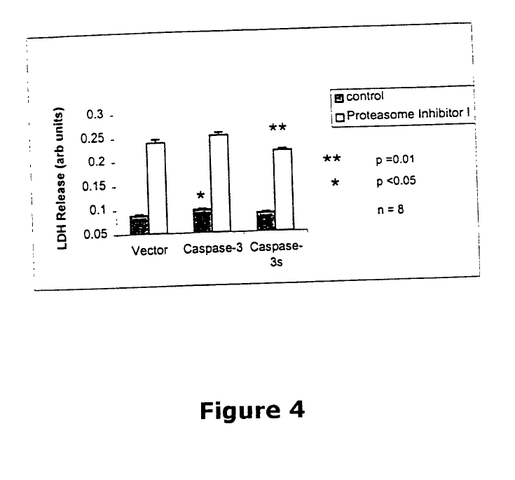

Data shown in FIG. 4 indicates that transfection of caspase-3 significantly increased apoptosis in the untreated condition (p<0.05). Upon exposure to proteasome inhibitor, both the vector control and caspase-3 transfected cells underwent substantial apoptosis. The increased apoptosis was significantly inhibited by caspase-3s transfection (p<0.01). These results demonstrate that caspase-3s has an apoptotic inhibitory activity.

### Example 7

The following experiments were performed to test the hypothesis that since caspase-3s lacks protease active site, it may not have apoptosis-inducing activity and instead might have antiapoptotic properties. To test this hypothesis, 293 cells were transfected with pcDNA3 vector, caspase-3 and caspase-3s cDNA constructs.

Transfection and Drug Treatment

Human embryonal kidney 293 cells were seeded 1×10⁵ into each well of a 24-well plate and transfected with caspase-3 and caspase-3s cDNA constructs by calcium phosphate method (Sun Y., Bian J., Wang Y., Jacobs C. Oncogene 1997;14:385-93;14: 385-393). Briefly, 500 nanogram of DNA was incubated in 0.25 M CaCl₂ solution for 10 minutes at room temperature. The mixture was then incubated with 1×HEPES buffer (pH 6.88) for 15 minutes at room temperature before being added to the culture medium. After 6 to 7 hours transfection, transfection medium was replaced with fresh Opti-MEMI medium (GIBCO) containing 5 μM of Proteasome Inhibitor I (Z-Ile-Glu(otBu)-Ala-Leu-CHO) (Calbiochem) and incubated for 16 hours. For staurosporine treatment, cells were allowed to grow for 24 hours in serum containing medium after transfection. Cells then incubated with 0.5 μM staurosporine (Calbiochem) in Opti-MEMI medium for 20 hours.

DNA Fragmentation Elisa

DNA fragmentation ELISA was performed using the Cell Death Detection ELISA kit (Boehringer Mannheim) following the instruction provided by the supplier. Briefly, after 16 hours treatment with IGAL (Proteasome Inhibitor I), 293 cells were lysed by incubation with 500 μL of cell lysis buffer for 2 hours at room temperature. The lysate were then centrifuged at 200×g for 10 minutes. Twenty microliter of the supernatant were transferred into the streptavidin coated microtiter plate for analysis using the reagents provided with the assay kit. Statistical analyses were performed with student t-test using Microsoft Excel analysis tools.

Western Blotting

Preparation of whole cell lysates and Western blotting were performed as previously described (McGinnis K. M., Gnegy M. E., Wang K. K. W. Endogenous bax translocation in SH-SY5Y human neuroblastoma cells and cerebellar granule neurons undergoing apoptosis. J. Neurochem. 1999;72:1899-1906; Huang Y., Domann F. E. Transcription factor AP-2 mRNA and DNA binding activity are constitutively expressed in SV40-immortalized but not normal human lung fibroblasts. Arch. Biochem. Biophys. 1999;364: 241-246). The blots were probed with antihuman caspase-3 p20 goat polyclonal antibody (Santa Cruz, CAT #sc-1226), antihuman caspase-3 rabbit polyclonal antibody (Pharmingen, CAT #65906E), antiflag monoclonal antibody (Sigma, CAT #F3165), or antihuman monoclonal PAPR antibody (Biomol Research Laboratories, Inc CAT #SA-250).

Results:

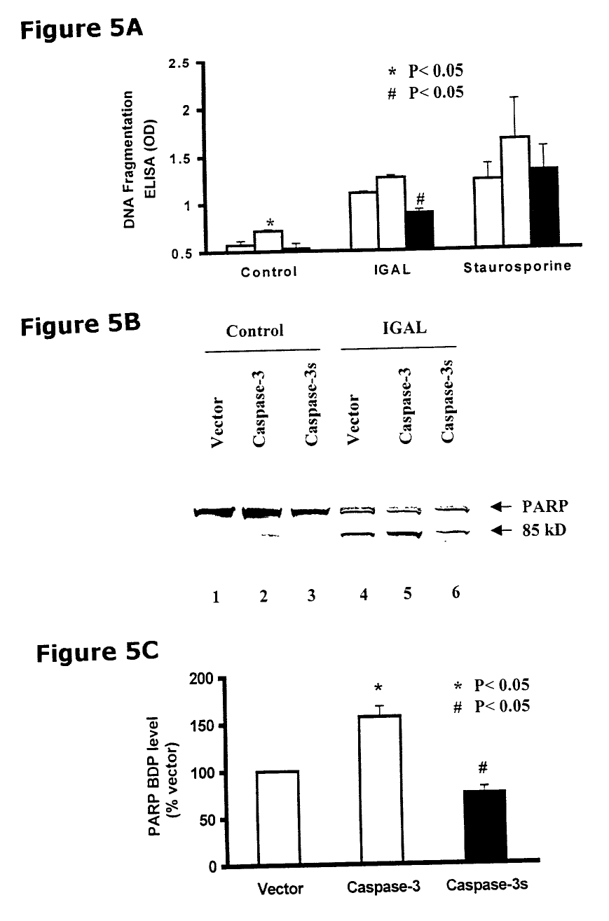

The transfected cells were challenged with two apoptosis-inducing agents: proteasome inhibitor Z-Ile-Glu(otBu)-Ala-Leu-CHO (IGAL) (Shinohara K., Tomioka M., Nakano H., Tone S., Ito H., Kawashima S. Apoptosis induction resulting from proteasome inhibition. Biochem. J. 1996;317:385-388; Drexler H. C. A. Activation of the cell death program by inhibition of proteasome function. Proc. Natl. Acad. Sci. USA 1997;94:855-860; Zhang X. M., Lin H., Chen C., Chen B. D. Biochem J. 1999;15:127-33; Qiu J. H., Asai A., Chi S., Saito N., Hamada H., Kirino T. Proteasome inhibitors induce cytochrome c-caspase-3-like protease-mediated apoptosis in cultured cortical neurons. J Neurosci. 2000;20:259-265) and protein kinase inhibitor staurosporine (Koh J. Y., Wie M. B., Gwag B. J., Sensi S. L., Canzoniero L. M., Demaro J., Csernansky C., Choi D. W. Staurosporine-induced neuronal apoptosis. Exp Neurol. 1995; 135:153-159; Posmantur R., McGinnis N. R., Gilbertsen R. and Wang K. K. W. Characterization of CPP32-like protease activity following apoptotic challenge in SH-SY5Y neuroblastoma cells. J. Neurochem. 1997;68:2328-2337). Overexpression of full-length capase-3 induced basal apoptosis of 293 cells IGAL as measured by DNA fragmentation ELISA, as expected. Overexpression of caspase-3s partially but significantly inhibited apoptosis induced by IGAL (FIG. 5A). On the other hand, overexpression of caspase-3 did not reduce stauropsorine-mediated 293 cell apoptosis.

It is known that cleavage of caspase-3 substrates, such as poly(ADP)ribose polymerase (PARP), is prerequisite for apoptosis. Therefore, the effects of caspase-3s overexpression on PARP cleavage in both control and IGAL treated 293 was examined, along with caspase-3 overexpression as positive control. Cleavage of PARP to an immunoreactive breakdown product (85 kDa) was measured by Western blot. Indeed, caspase-3s overexpression again partially but significantly inhibited the cleavage of PARP as compared to the vector control upon treatment with IGAL (lanes 6 vs. 4, FIGS. 5A and 5C). In contrast, overexpression of full-length caspase-3 increased basal PARP cleavage in the nontreated cells (lane 2), which was enhanced by IGAL treatment (lane 5 vs. 4, FIGS. 5B and 5C).

Proteasome inhibitor activates caspase-3 and stabilizes caspase-3s

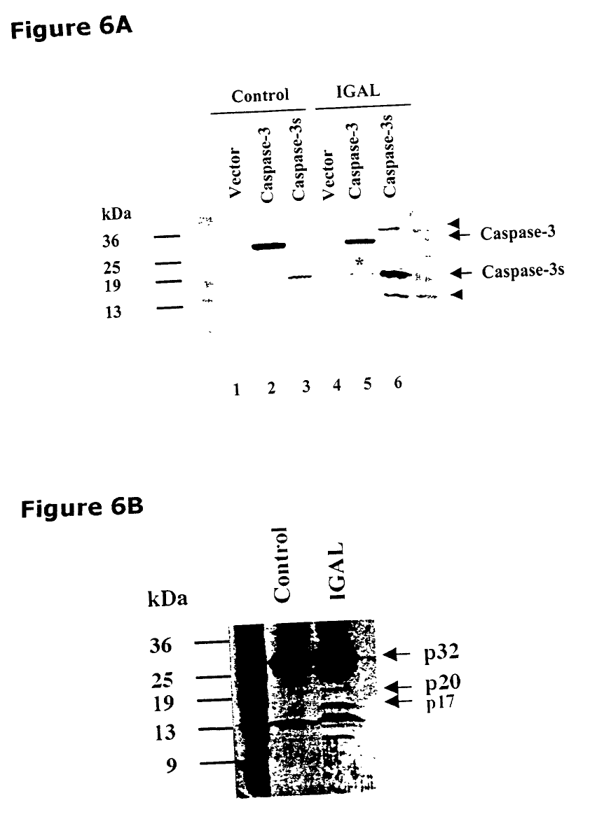

Proteasome appears to regulate caspase-3 potentially in two ways. Proteasome inhibition was reported by many groups to induce caspase-3 processing and activation, via a yet unknown mechanism (Zhang et al., Supra., 1999; Qiu et al., Supra., 2000). On the other hand, it was recently shown that full length caspase-3 was ubiquitinized by cIAP2 and potentially directing it toward active degradation by proteasome (Huang H. K., Joazeiro C. A., Bonfoco E., Kamada S., Leverson J. D., Hunter T. The inhibitor of apoptosis, cIAP2, functions as a ubiquitin-protein ligase and promotes in vitro monoubiquitination of caspases 3 and 7. J. Biol. Chem. 2000;275:26661-26664). Since caspase-3s was only expressed in low level in transfected 293 cells, we speculated that it might also be actively and selectively degraded by proteasome. To examine the effects of proteasome inhibitor on the expression of caspase-3 and caspase-3s, total cellular protein from transfected 293 cells treated with proteasome inhibitor IGAL were analyzed by Western blotting probed with the N-terminal antiflag antibody. Upon IGAL treatment, a decrease in the abundance of recombinant full length caspase-3 (32 kDa band) as well as the appearance of the 20 kDa activated form of caspase-3 (both containing the N-terminal flag-tag) were in fact observed (FIG. A, lanes 5 vs 2). Therefore, in this cell system, proteasome inhibition-mediated processing and loss of full-length capase-3 protein appears to be the predominant pathway. In contrast, we observed a dramatic increase in the amount of recombinant caspase-3s after IGAL treatment in transfected cells (detected by antiflag-tag antibody) (FIG. 6A, lanes 6 vs 3). This indicates that caspase-3s is a rapidly turnover protein, subjected to Ubiquitin-proteasome degradation. In addition, we reproducibly detected two additional bands with a size of 40 kDa and 14 kDa, respectively, after IGAL treatment (FIG. 6B, lane 6). The nature of these two bands is unknown at the present time. The 40 kDa band could be a homodimer of caspase-3s.

Evidence that the levels of endogenous caspase-3 and caspase-3s are regulated by proteasome degradation was also sought. Untransfected 293 cells were subjected to IGAL treatment and the cell samples were analyzed on Western blots probed with anticapase-3 antibody (Pharmingen). IGAL treatment led to the appearance of the 17 kDa activated caspase-3 (p17), while the levels of full-length caspase-3 (p32) remain the same (FIG. 6B). In parallel, a faint immunoreactive band of 20 kDa (p20) was detected only in the presence of IGAL treatment (FIG. 6B).

It will be clear that the present invention is well adapted to attain the ends and advantages mentioned as well as those inherent therein. While a presently preferred embodiment has been described for purposes of this disclosure, numerous changes may be made which will readily suggest themselves to those skilled in the art. Accordingly, all such modifications, changes and alternatives are encompassed in the spirit of the invention disclosed and as defined in the appended claims.

What is claimed is:

1. An isolated polynucleotide for caspase-3s comprising the nucleic acid sequence substantially similar to SEQ ID No. 1.

2. An isolated and purified polynucleotide that hybridizes to the DNA sequence for caspase-3s (SEQ ID No. 1) under high stringency hybridization conditions.

3. A vector comprising the polynucleotide of claim 1 in operative association with a nucleic acid regulatory sequence that controls the expression of the nucleic acid sequence in a host.

4. An isolated caspase-3s polypeptide comprising the amino acid sequence of SEQ ID No. 2.

5. A method of making a recombinant vector comprising inserting the isolated polynucleotide of claim 1 into a vector in both sense and antisense orientation.

6. A vector comprising the isolated polynucleotide of claim 1.

7. A host cell comprising the vector of claim 6.

8. An isolated polypeptide comprising a polypeptide member selected from the group consisting of:
(a) amino acids 1 to 182 of caspase-3s of SEQ ID No. 2;
(b) amino acids 28 to 182 of caspase-3s of SEQ ID No. 2; and
(c) amino acids 1 to 160 of caspase-3s of SEQ ID No. 2.

9. A method for inhibiting caspase activity in a cell, comprising:
introducing into the cell a nucleic acid sequence encoding a polynucleotide for caspase-3s comprising the sequence of SEQ ID No. 1.

10. A process according to claim 9 further comprising administering a proteasome inhibitor.

11. A process according to claim 10, wherein the proteasome inhibitor is proteasome inhibitor I (Z-Ile-Glu(otBu)-Ala-Leu-CHO).

12. A cDNA which codes for all or part of the caspase-3s polypeptide sequence of SEQ ID No. 2.

13. A diagnostic assay for detecting cells containing the caspase-3s splicing variant, comprising isolating total cell RNA, subjecting the RNA to reverse transcription-PCR amplification using primers from the isolated and purified polynucleotide sequence of claim 1 and determining whether the resulting PCR products coincide with the cDNA or the caspase-3s splicing variant.

14. A pharmaceutical composition comprising the substantially purified recombinant caspase-3s polypeptide of SEQ ID No. 2 and a pharmaceutically acceptable carrier.

15. A method for inhibiting growth of tumor cells comprising:
making a recombinant vector that expresses antisense recombinant caspase-3s polynucleotide of SEQ ID No. 1; and administering the recombinant vector to the tumor cells.

16. A method for inducing cytoprotection in a cell comprising:
administering to the cell a cytoprotective dose of a recombinant caspase-3s polypeptide of SEQ ID No. 2.

17. A method for inhibiting neuronal cell death, said method comprising:
introducing into a neuronal cell a nucleic acid sequence encoding a polyneucleotide for caspase-3s comprising the polynucleotide sequence of SEQ ID No. 1.

18. A method according to claim 17, further comprising administering a proteasome inhibitor.

19. A method according to claim 18, wherein the proteasome inhibitor is proteasome inhibitor I (Z-Ile-Glu(otBu)-Ala-Leu-CHO).

20. A method according to claim 18, wherein the proteasome inhibitor is coadministered with the polynucleotide encoding for caspase-3s.


SEQUENCE LISTING (5 sequences)

SEQ ID NO: 1 - DNA, 549 bp, Homo sapiens
atggagaacactgaaaactcagtggattcaaaatccattaaaaatttggaaccaaagatcatacatggaagcgaatcaatggactctggaatatccctggacaacagttataaaatggattatcctgagatgggtttatgtataataattaataataagaattttcataaaagcactggaatgacatctcggtctggtacagatgtcgatgcagcaaacctcagggaaacattcagaaacttgaaatatgaagtcaggaataaaaatgatcttacacgtgaagaaattgtggaattgatgcgtgatgtttctaaagaagatcacagcaaaaggagcagttttgtttgtgtgcttctgagccatggtgaagaaggaataatttttggaacaaatggacctgttgacctgaaaaaaataacaaactttttcagaggggatcgttgtagaagtctaactggaaaacccaaacttttcattattcaggttattattcttggcgaaattcaaaggatggctcctggttcatccagtcgctttgtgccatgctga

SEQ ID NO: 2 - protein, 182 aa, Homo sapiens
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQVIILGEIQRMAPGSSSRFVPC

SEQ ID NO: 3 - DNA, 63 bp, Homo sapiens
cgcggatccgccaccatggactacaaggacgacgatgacaaggagaacactgaaaactcagtg

SEQ ID NO: 4 - DNA, 40 bp, Homo sapiens
ccgctcgagttagtgataaaaatagagttcttttgtgagc

SEQ ID NO: 5 - DNA, 38 bp, Homo sapiens
ccgctcgagtcagcatggcacaaagcgactggatgaac